(19) United States
(12) Patent Application Publication
(10) Pub. No.: US 2024/0285889 A1
(43) Pub. Date: Aug. 29, 2024

(54) TUBE-WALL-FREE RIGHT-SIDED DOUBLE-LUMEN BRONCHIAL CATHETER

(71) Applicant: Jianwen ZHOU, Shanghai (CN)

(72) Inventor: Jianwen ZHOU, Shanghai (CN)

(21) Appl. No.: 18/573,843

(22) PCT Filed: Apr. 2, 2022
(86) PCT No.: PCT/CN2022/084957

§ 371 Date: Dec. 22, 2023

(30) Foreign Application Priority Data
Jul. 18, 2021 (CN) 202110810230.4
Jul. 18, 2021 (CN) 202121628179.7

(51) Int. Cl. A61M 16/04

(52) U.S. Cl. CPC A61M 16/0404; A61M 16/044; A61M 16/0459; A61M 16/0486; A61M 2210/1032; A61M 2210/1035; A61M 2210/1039

(57) ABSTRACT

A tube-wall-free right-sided double-lumen bronchial catheter, consisting of a tube-wall-free bronchial catheter body and a double-lumen ventilation catheter body. For the tube-wall-free bronchial catheter body, by means of a plurality of bronchial struts, an upper end is connected to the inner wall of a bronchial cuff and a lower end is connected to an annular opening, and no connection is present among the bronchial struts. When right lung single-lung ventilation is performed, the tube-wall-free bronchial catheter body enters a right main bronchus, the direction and position of the catheter body do not need to be adjusted, the tube-wall-free structure of the tube-wall-free bronchial catheter body can achieve complete opening of the upper lobe of the right lung, and the optimal ventilation effect of the right lung single-lung ventilation is ensured. Poor ventilation of the right upper lung is avoided, time consumption is shortened, airway injuries are reduced, and related complications are reduced.

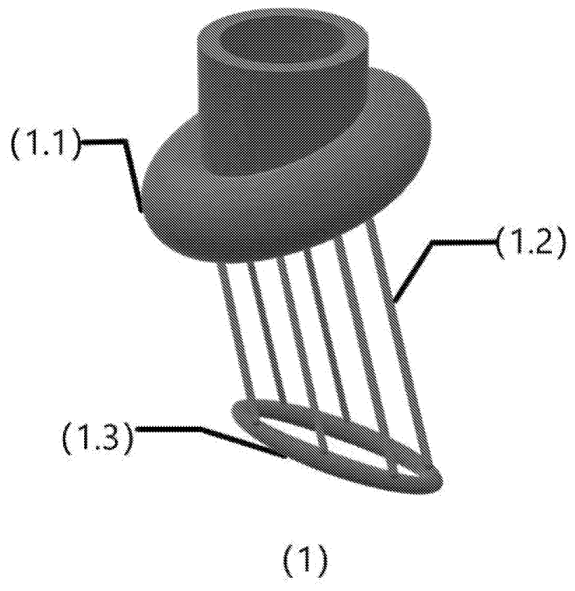

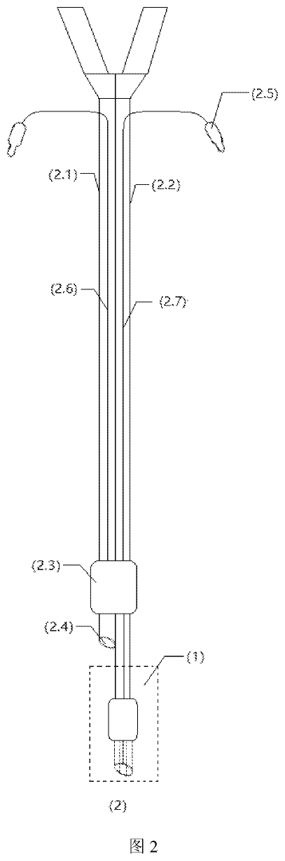

### TECHNICAL FIELD

The present invention relates to the technical field of medical equipment, specifically a tube-wall-free right-sided double-lumen bronchial catheter.

### Technical Backgrounds

The double-lumen bronchial catheter (DLT) can isolate the left lung from the other side, ensuring one-lung ventilation, providing good visual fields for the surgeon and preventing the healthy lung from being infected with blood and secretion of the affected lung, etc., which makes it the most frequently used ventilation tool in thoracic and cardiac surgery.

Currently, DLT frequently used are mainly categorized into left double-lumen bronchial catheter (LDLT) and right double-lumen bronchial catheter (RDLT). When using DLT for pulmonary isolation ventilation, it is preferable to place DLT into the non-operative side of the lung in order not to interfere with the surgical operation. To be specific, LDLT is used in right-sided thoracic surgery and RDLT is used in left-sided thoracic surgery.

Due to the anatomical structure of human airway, the left main bronchus is slender and long while the right one is relatively thick and short, and the bronchial opening of superior lobe of right lung is more variable and at a higher position. Therefore, sufficient ventilation can be achieved by using a LDLT for one-lung ventilation (OLV).

In comparison with LDLT, RDLT has side holes in the wall below the bronchial cuff to aid in ventilation of right upper lobe. In clinical practice, when using RDLT for OLV, anesthesiologists have to spend time adjusting the position and direction of RDLT to ensure adequate ventilation of upper lobe. However, poor catheter alignment, due to the high variability of the opening position of bronchial catheter in the upper lobe, and the blockage of the side holes with sputum, blood and other secretion may result in poor ventilation of the right lung, Which may cause oxygenation in the right lobe. It is more likely to cause a series of complications such as insufficient ventilation oxygenation, right upper lobe atelectasis, and severe imbalance of ventilation blood flow ratio.

Therefore, it is rather necessary to guarantee the highly efficient ventilation by using a RDLT that can ensure bilateral lung isolation and OLV of right lung.

### Contents of the Invention

The present invention breaks through today's technical bottleneck, and allows the RDLT to perform double-lung isolation, OLV, and at the same time ensure the ventilation effect to the greatest extent.

The tube-wall-free right-sided double-lumen bronchial catheter mainly include a tube-wall-free bronchial catheter body and double-lumen ventilation catheter body.

Further, the mentioned a tube-wall-free bronchial catheter body includes a bronchial cuff, several bronchial struts, and an annular opening.

Further, the mentioned double-lumen ventilation catheter body includes a main tracheal catheter, a right bronchial catheter, a main tracheal cuff, a main tracheal cuff inflation device, and a bronchial cuff inflation device, wherein a lower section of the right bronchial catheter is connected to a wall-less type bronchial catheter.

Further, the mentioned a tube-wall-free bronchial catheter body has a tube-wall-free structure of the bronchial catheter in the lower section of the bronchial cuff, and the tube-wall-free structure of the bronchial catheter is connected to the inner wall of the bronchial cuff by a plurality of bronchial struts connected to the inner wall of the bronchial cuff at an upper end and connected to the annular opening at a lower end, and the bronchial struts are connected to each other by the tube-wall-free structure of the bronchial struts.

Further, the mentioned double-lumen ventilation catheter body is a one-tube, two-lumen structure, with the left side being the main tracheal catheter and the right side being the right bronchial catheter.

Further, the main tracheal cuff bladder is in the lower section of the double-lumen ventilation catheter body, and it has a main tracheal catheter opening below the main tracheal cuff bladder, the opening being a beveled opening facing downwardly to the left.

Further, the main tracheal cuff inflation device is connected to the main tracheal cuff via a main tracheal cuff connection tube parallel to the dual lumen ventilation catheter.

Further, the bronchial cuff inflation device is connected to the bronchial cuff via a bronchial cuff connection tube parallel to the dual lumen ventilation catheter.

In comparison with current techniques the present invention breaks through the constraints of the “tube” in the tracheal catheter, and “wall-less” design in bronchial catheter maximally guarantees the normal ventilation of the upper lobe of the right lung and ensure optimal ventilation of the right lung in single-lung ventilation, various measures are taken to prevent issues such as misalignment of side holes, obstruction of side holes by secretions, and blocking of the tracheal opening by the tube wall. These measures aim to minimize ventilation impairment in the right upper lung, ensuring proper airflow and reducing the risk of complications associated with inadequate ventilation. Additionally, by minimizing the time required for adjusting the tube and the potential for airway injuries, this approach contributes to an overall improvement in the efficiency and safety of single-lung ventilation.

### ATTACHED PICTURES

Pic. 1 shows a schematic diagram of the structure of a tube-wall-free bronchial catheter body;

Pic. 2 shows a schematic diagram of the overall structure of a tube-wall-free right-sided double-lumen bronchial catheter.

In attached drawings: 1.1 bronchial cuff; 1.2 bronchial strut; 1.3 annular opening; 2.1 main tracheal catheter; 2.2 right bronchial catheter; 2.3 main tracheal cuff; 2.4 a main tracheal catheter opening; 2.5 bronchial cuff inflator; 2.6 main tracheal cuff connection tube 2.7 bronchial cuff connection tube

### SPECIFIC EMBODIMENTS

The present invention will be further described below with attached drawings, the description doesn't constitute a limitation of the present invention.

Referring to the pic 1 and 2, the tube-wall-free right-sided double-lumen bronchial catheter tube consists primarily of the tube-wall-free bronchial catheter body 1 and double-lumen ventilation catheter body 2. The tube-wall-free bronchial catheter body 1 comprises the bronchial cuff 1.1, several bronchial struts 1.2 and the annular opening 1.3. The bronchial catheter of the lower section of the bronchial cuff 1.1 in the tubeless wall type bronchial catheter 1 has no tubular wall structure, and the bronchial catheter of the tubeless wall structure is connected to the inner wall of the bronchial cuff 1.1 via the upper end of a plurality of bronchial struts 1.2, the lower end of a plurality of bronchial struts 1.2 connected to the annular opening 1.3, bronchial struts 1.2 connected to each other without wall structure. The mentioned double-lumen ventilation catheter body 2 consists of the main tracheal catheter 2.1, the right bronchial catheter 2.2, the main tracheal cuff 2.3, the main tracheal catheter opening 2.4, the main tracheal cuff inflation device, the bronchial cuff inflation device 2.5, among them, the lower end of the right bronchial catheter connected to the tube-wall-free bronchial catheter body 1. Double-lumen ventilation catheter body 2 is a tube with two-lumen structure, with the left side being the main tracheal catheter 2.1 and the right side being the right bronchial catheter 2.2. The main tracheal cuff 2.3 is in the lower section of the double-lumen ventilation catheter body 2, and below the main tracheal cuff 2.3 is the main tracheal catheter opening 2.4, which is a beveled opening facing downward to the left. The main tracheal cuff inflation device is connected to the main tracheal cuff 2.3 via the main tracheal cuff connection tube 2.6 parallel to the double-lumen ventilation catheter body 2. The bronchial cuff inflation device 2.5 is connected to the bronchial cuff 1.1 via the bronchial cuff connection tube 2.7 parallel to the double-lumen ventilation catheter body 2.

The main tracheal cuff connection tube 2.6 and the bronchial cuff connection tube 2.7 are thin tubes.

In the specific implementation of the present invention, when the a tube-wall-free bronchial catheter body enters the right main bronchus and the bronchial cuff is flush with the level of the tracheal bulge of the bronchial cuff, the main tracheal catheter cuff and the bronchial cuff are inflated, and during the process of OLV, there is no need for adjusting the catheter direction and position, and the tube-wall-free structure of the bronchial catheter can realize the complete opening of the right upper lobe of the lung to guarantee the optimal ventilating effect of OLV.

On the basis of the traditional RDLT, the present invention abandons the previous structure of the right bronchial catheter, breaking through the barrier of the “tube”, and the right bronchial catheter adopts the structure of the tubeless wall, which changes the method of relying on the wall, the lumen, and the structure of the side hole in the traditional catheter to carry out ventilation, and radically solves the problem of misalignment, secretion blockage, and the problem of the side hole, which are the most common problems of the right lung, and the most common problems of the right lung. It can fundamentally solve the problem of poor ventilation caused by misalignment of side holes, obstruction of side holes by secretions, and blocking of the tracheal opening by the tube wall, which not only saves the anesthesiologist's time for adjusting the positioning, but also maximally protects the patient's OLV effect and avoids various complications caused by poor ventilation due to the defect of the catheter. The design of the present invention of the tubeless wall bronchial catheter is simple, but it is easy to manufacture and has remarkable effects and strong Clinical practical value. If it can be popularized and applied, it will play a significant clinical value.

The foregoing is only a preferred embodiment of the present invention model, and it should be noted that a number of improvements and additions, without departing from the method of the present invention model, can be made by ordinary technical personnel, which shall also be considered to be within the scope of protection of the present invention model.

1. A tube-wall-free right-sided double-lumen bronchial catheter is characterized by the tube-wall-free bronchial catheter body and the double-lumen ventilation catheter body.

2. The tube-wall-free right-sided double-lumen bronchial catheter mentioned in claim 1 is characterized by the mentioned tube-wall-free bronchial catheter body consisting of the bronchial cuff, several bronchial struts and the annular opening.

3. The tube-wall-free right-sided double-lumen bronchial catheter mentioned in claim 2 is characterized by the mentioned double-lumen ventilation catheter consisting of the main tracheal catheter, the right bronchial catheter, the main tracheal cuff, the main tracheal cuff inflation device, the bronchial cuff inflation device, among them, the lower end of the right bronchial catheter connected to the tubeless wall type bronchial catheter.

4. The tube-wall-free right-sided double-lumen bronchial catheter mentioned in claim 2 is characterized by the mentioned bronchial catheter of the lower section of the bronchial cuff in the tube-wall-free bronchial catheter body has no tubular wall structure, and the bronchial catheter of the tubeless wall structure is connected to the inner wall of the bronchial cuff via the upper end of several bronchial struts, the lower end of several bronchial struts connected to the annular opening, bronchial struts connected to each other without wall structure.

5. The tube-wall-free right-sided double-lumen bronchial catheter mentioned in claim 3 is characterized by the mentioned double-lumen ventilation catheter body is a tube with two-lumen structure, with the left side being the main tracheal catheter and the right side being the right bronchial catheter.

6. The tube-wall-free right-sided double-lumen bronchial catheter mentioned in claim 3 is characterized by the mentioned the main tracheal cuff is in the lower section of the double-lumen ventilation catheter body, and below the main tracheal cuff is the main tracheal catheter opening, which is a beveled opening facing downward to the left.

7. The tube-wall-free right-sided double-lumen bronchial catheter mentioned in claim 3 is characterized by the mentioned the main tracheal cuff inflation device is connected to the main tracheal cuff via the main tracheal cuff connection tube parallel to the double-lumen ventilation catheter body.

8. The tube-wall-free right-sided double-lumen bronchial catheter mentioned in claim 3 is characterized by the mentioned the bronchial cuff inflation device is connected to the bronchial cuff via the bronchial cuff connection tube parallel to the Double-lumen ventilation catheter body.
